(19) United States
(12) Patent Application Publication
(10) Pub. No.: US 2006/0079956 A1
(43) Pub. Date: Apr. 13, 2006

(54) Bifurcation stent with crushable end and method for delivery of a stent to a bifurcation

(75) Inventors: Neal L. Eigler, Pacific Palisades, CA (US); Frank Litvack, Los Angeles, CA (US); John F. Shanley, Redwood City, CA (US); Stephen Hunter Diaz, Palo Alto, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/200,765

(22) Filed: Aug. 10, 2005

Related U.S. Application Data
(60) Provisional application No. 60/610,279, filed on Sep. 15, 2004.

(51) Int. Cl. A61F 2/06

(52) U.S. Cl. 623/1.35

(57) ABSTRACT

A bifurcation stent includes a first end which is deformable or crushable at a lower force than a second end. The crushable first end and more rigid second end of the bifurcation stent allow one end of the stent to remain expanded in tissue supporting configuration in a side branch of a vessel bifurcation while the other end is easily crushed against the side wall of the main vessel into which it extends. A method of supporting a bifurcated body lumen with the bifurcation stent involves delivering the bifurcation stent in an unexpanded configuration to a bifurcation in a body lumen, positioning the bifurcation stent with the distal portion substantially within a side branch vessel of the bifurcation and the proximal crushable portion substantially within the main vessel, expanding the bifurcation stent, and expanding a main vessel stent along side the bifurcation stent and thereby crushing at least a portion of the crushable proximal portion of the bifurcation stent against the main vessel wall.

### RELATED APPLICATIONS

This application claims priority to U.S. Provisional Patent Application Ser. No. 60/610,279, filed Sep. 15, 2004, the entire contents of which are incorporated herein by reference.

### BACKGROUND

In the past, permanent or biodegradable devices have been developed for implantation within a body passageway to maintain patency of the passageway. These devices are typically introduced percutaneously, and transported transluminally until positioned at a desired location within the body passageway. The devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Frequently, the area to be supported by such devices is located at or near the junction of two or more lumens, called a bifurcation. In coronary angioplasty procedures, for example, it has been estimated that 15% to 20% of cases involve reinforcing the area at the junction of two arteries. Conventional stent implantation at such a junction results in at least partial blockage of the branch vessel, affecting blood flow and impeding access to the branch vessel for further angioplasty procedures.

One known technique for treating bifurcations generally deliver a mesh stent into the vessel and position the device over the bifurcation. According to the known methods, a surgeon then attempts to create one or more branch lumen access holes by inserting a balloon through the sidewall of the mesh device, and then inflating the balloon to simply push the local features of the mesh aside. These techniques are inherently random in nature: the exact point of expansion in the device lattice cannot be predicted, and the device may or may not expand satisfactorily at that point. Tissue support provided by these known techniques for treating bifurcated arteries is similarly unpredictable. In addition, the effectiveness of such procedures is limited because many mesh devices are unable to accommodate such expansion at random locations in the device structure. Further, prior art stent delivery systems are unable to accurately position specific device features over the branch vessel opening.

Another method for deploying a stent in a bifurcating vessel is described in International Application WO98/19628. According to this method, a main stent having a substantially circular side opening and a flared stent having a flared end are used together to treat a bifurcating vessel in a two step process. In a first step, the main stent is positioned using an inflatable balloon catheter in the interior of the main stent and a stabilizing catheter extending through the side opening of the stent. The stabilizing catheter is used to place the side opening in the main stent at the opening to the branch vessel. The main stent is then expanded and the flared stent is inserted through the side opening into the vessel bifurcation. One drawback of this method is the difficulty in positioning the side opening of the main stent at a proper longitudinal and radial position at the vessel bifurcation. Another drawback of this system is the flared stent which is difficult to form and position, and may tend to protrude into the blood stream causing thrombosis.

One current method of treating bifurcations is called the crush method. In this method, a first stent is placed into the branch vessel extending from the branch vessel into the main vessel and a second stent is placed in the main vessel across the bifurcation. The first stent is deployed in the branch vessel and the first balloon is withdrawn. The second stent is then deployed in the main vessel crushing a proximal portion of the first stent against the main vessel wall. This crush method appears to provide generally successful results supporting both the main vessel and the branch vessel. However, in cases where the proximal end of the first stent is not completely crushed there may be a tendency to protrude into the bloodstream providing an opportunity for thrombosis. Further, the act of crushing the first stent can tend to pull a portion of the stent away from the branch vessel it supports right at the vessel junction where support is needed most.

In view of the drawbacks of the prior art bifurcated tissue supporting systems, it would be advantageous to have a bifurcation stent system and a bifurcation stent delivery system capable of providing superior support with minimal resistance to flow into the branch vessel.

### SUMMARY OF THE INVENTION

The present invention relates to a method of supporting a bifurcated body lumen comprising the steps of delivering an bifurcation stent in an unexpanded configuration to a bifurcation in a body lumen, the bifurcation stent having a distal portion and a crushable proximal portion which is deformable at a lower force than the distal portion, positioning the bifurcation stent with the distal portion substantially within a side branch vessel of the bifurcation and the proximal portion substantially within the main vessel, expanding the bifurcation stent into a seated arrangement in the side branch vessel, and expanding a main vessel stent along side the bifurcation stent and thereby crushing at least a portion of the crushable proximal portion of the bifurcation stent against the main vessel wall.

In accordance with one aspect of the invention, a method of supporting a bifurcated body lumen comprises the steps of delivering a pre-crushed stent into a side branch vessel of a bifurcation, the pre-crushed stent having a distal tubular tissue supporting portion and a proximal crushed portion, arranging the pre-crushed stent with the distal tubular tissue supporting portion substantially within a side branch vessel of the bifurcation and the proximal crushed portion extending into a main vessel of the bifurcation and expanding the distal tubular tissue supporting portion of the stent within the side branch.

In accordance with another aspect of the invention, a pre-crushed stent comprises a continuous tubular body expandable from a delivery configuration to an expanded tissue supporting configuration, the body at the delivery configuration having a first tubular tissue supporting segment and a second crushed portion connected to the first tubular portion.

In accordance with an additional aspect of the invention a stent and delivery system is comprised of a pre-crushed stent comprising a continuous tubular body expandable from a delivery configuration to an expanded tissue supporting configuration, the body at the delivery configuration having a first tubular tissue supporting portion and a second crushed portion connected to the first tubular portion and a balloon catheter comprising a balloon positioned within the first tubular tissue supporting portion of the pre-crushed stent.

In accordance with a further aspect of the invention a method of delivering a stent to a bifurcated body lumen comprises the steps of delivering an expandable stent in an unexpanded configuration to a bifurcation in a body lumen, the bifurcation having a main vessel and a side branch vessel, at least partially expanding a proximal portion of the stent, advancing a distal end of the stent into the side branch vessel of the bifurcation until a junction between the expanded proximal portion and an unexpanded distal portion of the stent is seated into the opening of the side branch vessel and expanding the distal portion of the stent in the side branch vessel.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is an enlarged side view of a portion of the stent of FIG. 1 showing a crushable end portion of the stent.

FIG. 3A is a schematic side view of a blood vessel bifurcation and a stenting system with a bifurcation stent having a crushable end.

FIG. 3B is a schematic side view of the system of FIG. 3A with a partially expanded crushable end and the bifurcation stent advanced to seat in the bifurcation.

FIG. 3C is a schematic side view of the system of FIG. 3A with the bifurcation stent fully expanded in the side branch.

FIG. 3D is a schematic side view of the system of FIG. 3A with the main vessel stent fully expanded and the crushable end of the bifurcation stent crushed.

FIG. 4 is a schematic side view of an expanded pre-crushed stent for bifurcations.

FIG. 5A is a schematic side view of the pre-crushed stent of FIG. 4 mounted on a balloon catheter in an unexpanded configuration.

FIG. 5B is a schematic side view of the pre-crushed stent of FIG. 4 mounted on a balloon catheter and expanded.

FIG. 6A is a schematic side view of a blood vessel bifurcation and a stenting system with a bifurcation stent expanded in the side branch and a pre-crushed end in the main vessel.

FIG. 6B is a schematic side view of the system of FIG. 6A with the main vessel stent fully expanded.

### DETAILED DESCRIPTION

The term “crush” or “crushed” as used herein refers to the collapsing of one or both opposite sides of a tubular member so that the opposite sides contact or nearly contact one another.

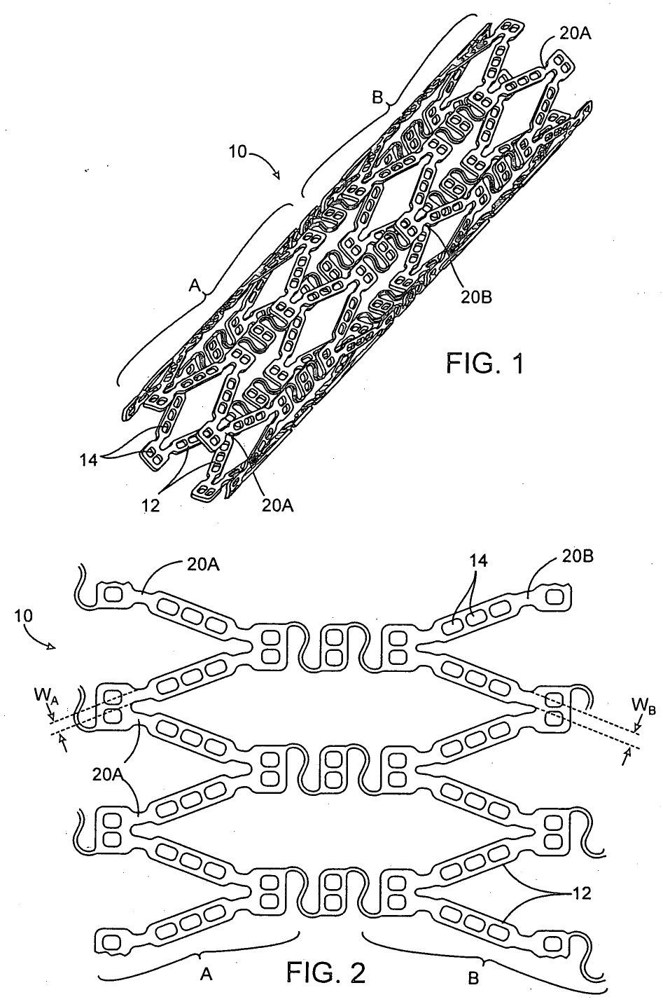

FIGS. 1 and 2 illustrate one example of a bifurcation stent 10 having a first end A which is deformable or crushable at a lower force than a second end B. The crushable first end A and more rigid second end B of the bifurcation stent allow one end of the stent to remain expanded in tissue supporting configuration in a side branch of a vessel bifurcation while the other end is easily crushed against the side wall of the main vessel into which it extends.

The stent 10 in the example of FIGS. 1 and 2 has a plurality of struts 12 interconnected by a plurality of ductile hinges 20A and 20B. Upon expansion or compression of the stent, the ductile hinges 20A and 20B plastically deform while the struts are not plastically deformed. The ductile hinges 20A in the crushable end A of the stent 10 have a width W_{A} which is smaller than a width W_{B} of the hinges 20B in the side branch supporting end B of the stent. The width of the hinges 20A and 20B is measured in a direction substantially perpendicular to a longitudinal axis of the adjacent struts or substantially perpendicular to the longitudinal axis of the stent when the stent is in an unexpanded configuration. This difference in width of the hinges provides a crushable end A which is expandable at a lower force and is more easily crushed (crushable at a lower force) than the second end B with wider hinges.

The crushable end A of the bifurcation stent 10 can also be provided by varying other dimensions or materials of the stent. For example, the hinge thickness or hinge material may be varied to achieve the crushable end. The stent can also be a stent without hinges and the properties of the deformable struts themselves can be varied to achieve the crushable end. For example, the strut thickness, strut width, or strut material can be varied to create the crushable end. Alternatively, the strut arrangement, length, number, or shape of struts can be changed to create the crushable end A.

In one example, the crushable end is formed by decreasing the radial thickness of the entire stent at one end resulting in a thin walled crushable end and a thick walled vessel supporting end. The thin walled crushable end can be formed by electropolishing, chemical etching, or the like.

In one example of a chemical etching process, the entire stent is coated in photo resist, such as by dipping. The photo resist on the inner or outer surface of the stent is removed to allow radial etching or thinning of the stent walls without etching the side surfaces of the struts or the inner surfaces of the holes. The selected removal of photo resist can be performed by inserting a pin inside the stent in the crushable end only. The pin fits into the stent blocking the passage of light to the interior surfaces of the crushable end. The entire stent is then exposed to UV light which cross links the exposed photo resist preventing it from being removed by a subsequent solvent. The pin is then removed and a solvent is used to remove the uncrosslinked photo resist from the interior surface of the crushable end. The stent is electro polished to thin the crushable end to a desired thickness and then the photo resist is removed from a remainder of the stent with a solvent.

The stent 10 of FIGS. 1 and 2 is illustrated with a plurality of openings 14 for providing a beneficial agent, such as an antirestenotic drug. It should be understood that these openings may be omitted when no drug is desired. Alternatively, the stent 10 can be coated or otherwise impregnated with a beneficial agent.

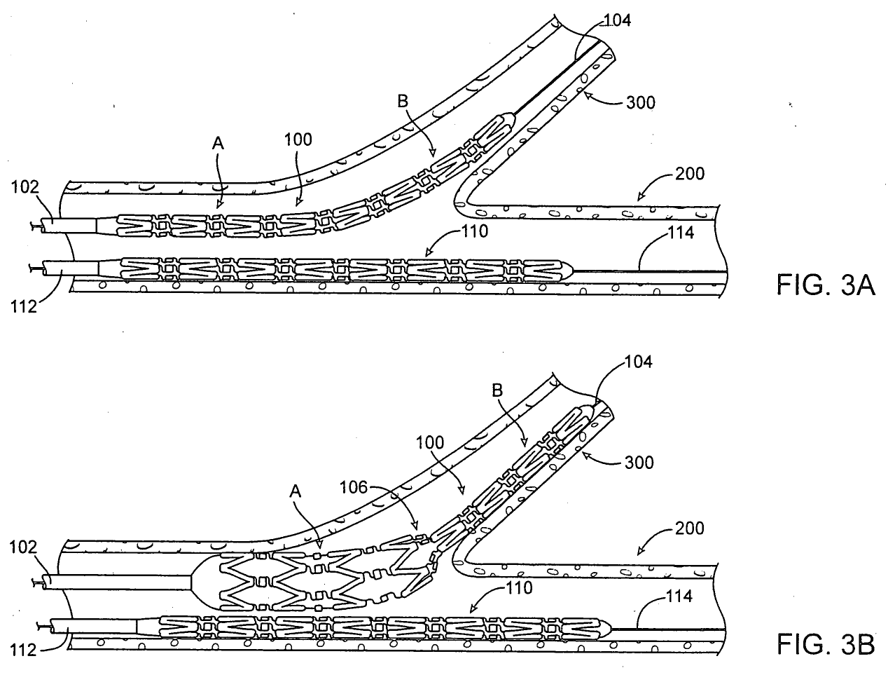

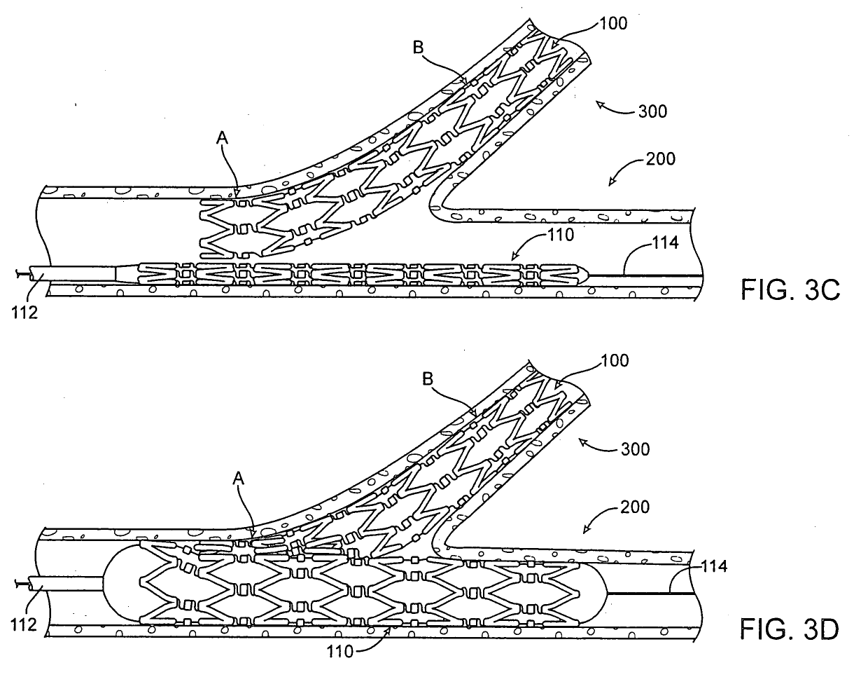

FIGS. 3A-3D illustrate a stenting system and a method of stenting a bifurcation with a first stent 100 having a crushable end A as described above and a second stent 110 without the crushable end. FIGS. 3A-3D show a blood vessel bifurcation with a main vessel 200 and a side branch vessel 300 extending from the main vessel to form a Y shape. As shown in FIG. 3A, the bifurcation stent 100 is advanced into the vasculature to the location of the bifurcation in a known manner using a first balloon catheter 102 and a first guidewire 104. The second stent 110 or main vessel stent is delivered with a second balloon catheter 112 and a second guidewire 114.

FIG. 3B illustrates a crushable end A of the bifurcation stent 100 which has been partially expanded by expansion of the first balloon at a first pressure. Due to reduced radial strength of the crushable end, the crushable end or proximal end of the stent 100 will expand at least partially upon application of the first pressure, while the distal end B of the stent is not expanded.

According to one embodiment, the bifurcation stent 100 can be advanced slightly with the crushable end A partially expanded so that the stent is seated into the side branch opening of the bifurcation as shown in FIG. 3B. The seating can be determined by the resistance to pushing felt when contact is made. In this way the transition area 106 between the crushable proximal end A and the distal end B of the bifurcation stent 100 can be accurately positioned at the side branch opening. To prevent damage to the vessel walls during advancement, the stent should be expanded to a diameter less than the inner diameter of the main vessel, and preferably at least 10% less than the diameter of the main vessel.

Alternatively, marker bands or other visualizing means can be used to position the transition area 106 at the side branch opening. When such known visualization techniques are used, the step of partial inflation of FIG. 3B can be omitted and the bifurcation stent 100 can be positioned by visualization prior to balloon inflation.

FIG. 3C illustrates the bifurcation stent 100 fully expanded in the side branch vessel 300 with the crushable proximal end A extending into the main vessel 200. The stent 100 has been expanded by inflation of the balloon catheter 102, shown in FIGS. 3A and 3B, to a second pressure higher than the pressure used to achieve the partial expansion of the proximal end shown in FIG. 3B. In the expanded configuration, the bifurcation stent 100 supports the walls of the side branch 300 distal to the bifurcation and extends alongside the second stent 110 in the main vessel 200.

FIG. 3D shows the expansion of the main vessel stent 110 by the balloon catheter 112. This expansion crushes the crushable proximal end A of the bifurcation stent 100 against the wall of the vessel. The force required to crush the crushable proximal end can be about 80% or less than the force required to crush the distal end B. In one example, the force required to crush the crushable end A is 60% or less of the force required to crush the distal end B.

As shown in FIG. 3D, the distal end B of the bifurcation stent 100 continues to support the side branch vessel 300. Blood flow into the side branch vessel 300 passes through the openings between the struts in the main vessel stent 110 and in the bifurcation stent 100. The location of the stent struts across the opening to the side branch vessel 300 generally has an insignificant effect on the blood flow into the side branch vessel. In some instances, it may be desirable to further open one or more of the openings between the struts at the side branch vessel opening by inserting a balloon catheter between the struts and expanding the balloon to increase the spacing between the struts.

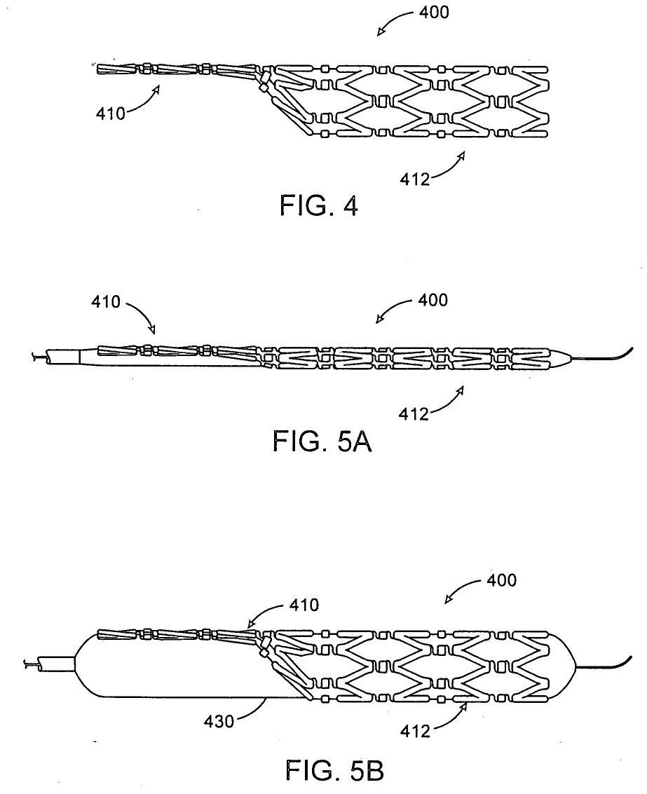

FIGS. 4 and 5 illustrate an alternative embodiment of a pre-crushed bifurcation stent 400 which has a pre-crushed end 410 for use in stenting a bifurcation. The stent 400 includes an expandable end 412 formed of a plurality of interconnected struts which form a substantially cylindrical end. The expandable cylindrical end 412 is connected to the crushed end 410 by the plurality of struts. The bifurcation stent 400 can be formed from any know stent by crushing one end of the stent prior to delivery. The pre-crushed end 410 may have the same or a different structure than the expandable end 412. For example, the pre-crushed end may have a reduced number of struts.

As shown in FIGS. 5A and 5B, the pre-crushed bifurcation stent 400 is mounted on a balloon catheter 430 with the balloon positioned within the expandable cylindrical end 412 of the catheter and the balloon positioned along side of the pre-crushed end 410. This configuration is achieved by passing the balloon catheter 430 through an opening between the struts of the stent 400. The crushed end 410 is flattened and laid along the outside of the balloon in a relatively flat configuration. The arrangement of the catheter with the balloon extending through a side hole in the stent 400 provides the additional benefit of expanding a cell at the side branch vessel during expansion of the stent 400. This expansion of a cell at the side branch vessel opening reduces the number of struts traversing the opening, thus improving blood flow.

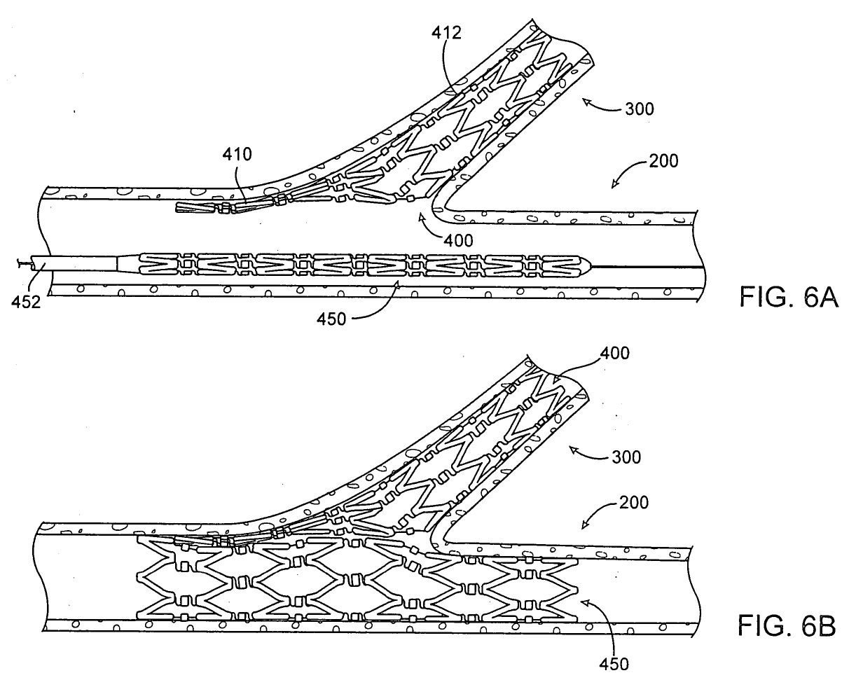

FIGS. 6A and 6B illustrate a stenting system and method of stenting a bifurcation with the pre-crushed stent 400 of FIGS. 4, 5A, and 5B. As shown in FIG. 6A, the pre-crushed stent 400 is delivered to the bifurcation by a balloon catheter and positioned with the distal expandable end 412 within the side branch lumen 300. The pre-crushed stent 400 is arranged such that the pre-crushed end 410 is located at a proximal side of the side branch opening by rotation of the catheter shaft. The proper stent orientation can be confirmed visually by known methods. In the event that the stent is not visible, radiopaque marker bands or other markers may be used in a known manner. Although the preferred orientation of the pre-crushed end 410 is directly proximal of the side opening as shown in FIGS. 6A and 6B, a side oriented pre-crushed end can also be used successfully.

After the pre-crushed stent 400 has been positioned and oriented, the stent 400 is then expanded by the balloon catheter so that the pre-crushed end 410 extends along the side wall of the main vessel. The main vessel stent 450 can be advanced to the bifurcation site by the catheter 452 either before of after the expansion of the pre-crushed stent 400. The main vessel stent 450 is expanded, as shown in FIG. 6B, to support the main vessel lumen at the bifurcation and traps the pre-crushed end 410 of the bifurcation stent 400 against the main vessel wall. The resulting expanded two stent arrangement for supporting the bifurcation as shown in FIG. 6B is similar to that achieved in FIG. 3D.

In the embodiments described above, the main vessel stents can be of the same general configuration as the side branch vessel stents. Alternatively, different sizes, shapes and configurations can be used for the main vessel stents and the crushable or pre-crushed stents. In one embodiment, the main vessel stent is longer than the side branch stent to ensure that the entire proximal end of the side branch stent is crushed and flattened against the main vessel wall.

According to one aspect of the invention, the stents described above can be drug delivery stents. When drug delivery is used in combination with the crushable bifurcation stents described herein, the crushable stent can contain no drug or less drug on the crushable or pre-crushed end to prevent double dosing of the vessel wall at the location of the crushed proximal end. Further increased drug concentration can be provided at particularly problematic regions. For example the area of the opening of the side branch vessel is a particularly problematic region of the bifurcation and can receive more drug by increasing drug concentration in a central region of the crushable stent.

When the stent includes the drug in openings, the increased drug concentration can be provided by increasing the dose per opening, by increasing the number of openings, or by increasing a size of the openings.

While the invention has been described in detail with reference to the preferred embodiments thereof, it will be apparent to one skilled in the art that various changes and modifications can be made and equivalents employed, without departing from the present invention.

1. A method of supporting a bifurcated body lumen, the method comprising:
delivering an bifurcation stent in an unexpanded configuration to a bifurcation in a body lumen, the bifurcation stent having a distal portion and a crushable proximal portion which is deformable at a lower force than the distal portion;
positioning the bifurcation stent with the distal portion substantially within a side branch vessel of the bifurcation and the proximal portion substantially within the main vessel;
expanding the bifurcation stent into a seated arrangement in the side branch vessel;
expanding a main vessel stent along side the bifurcation stent and thereby crushing at least a portion of the crushable proximal portion of the bifurcation stent against the main vessel wall.

2. The method of claim 1, further comprising locating the unexpanded bifurcation stent in the side branch vessel by a visual indication.

3. The method of claim 1, further comprising locating the unexpanded bifurcation stent in the side branch vessel by a tactile indication.

4. The method of claim 1, wherein the crushable proximal portion of the bifurcation stent is formed with deformable portions having a smaller width than deformable portions of the distal portion of the device.

5. The method of claim 1, wherein the crushable proximal portion of the stent is formed with deformable portions having a smaller thickness than deformable portions of the distal portion of the device.

6. The method of claim 1, wherein the crushable proximal portion of the stent is formed of a different material than the distal portion allowing the crushable proximal portion to be deformed more easily than the distal portion.

7. The method of claim 1, wherein the crushable proximal portion of the stent is formed of a different strut configuration than the distal portion allowing the crushable proximal portion to be deformed more easily than the distal portion.

8. A method of supporting a bifurcated body lumen, the method comprising:
delivering a pre-crushed stent into a side branch vessel of a bifurcation, the pre-crushed stent having a distal tubular tissue supporting portion and a proximal crushed portion;
arranging the pre-crushed stent with the distal tubular tissue supporting portion substantially within a side branch vessel of the bifurcation and the proximal crushed portion extending into a main vessel of the bifurcation; and
expanding the distal tubular tissue supporting portion of the stent within the side branch.

9. The method of claim 8, further comprising delivering a main vessel stent to the main vessel adjacent an opening of the side branch vessel, and expanding the main vessel stent into contact with the crushed portion of the pre-crushed stent.

10. The method of claim 8, wherein the pre-crushed stent is delivered on a balloon catheter having a balloon positioned within the distal tubular portion of the pre-crushed stent.

11. The method of claim 10, wherein the balloon catheter passes through a side hole in the pre-crushed stent and along an outside surface of the crushed portion of the pre-crushed stent.

12. The method of claim 11, wherein the balloon catheter aligns the side hole in the pre-crushed stent with the opening into the side branch vessel of the bifurcation.

13. A pre-crushed stent comprising a continuous tubular body expandable from a delivery configuration to an expanded tissue supporting configuration, the body at the delivery configuration having a first tubular tissue supporting segment and a second crushed portion connected to the first tubular portion.

14. The pre-crushed stent of claim 13, wherein the second crushed portion has a strut arrangement which is the same as the first tubular portion.

15. The pre-crushed stent of claim 13, wherein the second crushed portion has a strut arrangement which is different from the first tubular portion.

16. The pre-crushed stent of claim 13, wherein the second crushed portion is crushed such that a first side of the tube is in contact with a second side of the tube substantially eliminating any tube lumen at the second portion.

17. The pre-crushed stent of claim 13, wherein the first tubular portion includes a drug.

18. A stent and delivery system comprising:
a pre-crushed stent comprising a continuous tubular body expandable from a delivery configuration to an expanded tissue supporting configuration, the body at the delivery configuration having a first tubular tissue supporting portion and a second crushed portion connected to the first tubular portion; and
a balloon catheter comprising a balloon positioned within the first tubular tissue supporting portion of the pre-crushed stent.

19. The system of claim 18, wherein the balloon catheter extends outside of the second crushed portion.

20. The system of claim 18, wherein the second crushed portion has a strut arrangement which is the same as the first tubular portion.

21. The system of claim 18, wherein the second crushed portion has a strut arrangement which is different from the first tubular portion.

22. The system of claim 18, wherein the second crushed portion is crushed such that a first side of the tubular portion is in contact with a second side of the tubular portion substantially eliminating any tube lumen at the second crushed portion.

23. The system of claim 18, wherein the balloon catheter extends through a side opening between struts on the circumferential surface of the stent.

24. The system of claim 18, wherein the pre-crushed stent includes a drug.

25. A method of delivering a stent to a bifurcated body lumen, the method comprising:
delivering an expandable stent in an unexpanded configuration to a bifurcation in a body lumen, the bifurcation having a main vessel and a side branch vessel;
at least partially expanding a proximal portion of the stent;
advancing a distal end of the stent into the side branch vessel of the bifurcation until a junction between the expanded proximal portion and an unexpanded distal portion of the stent is seated into the opening of the side branch vessel; and
expanding the distal portion of the stent in the side branch vessel.

26. The method of claim 25, wherein the proximal portion of the stent is expandable by application of a first force applied by inflating a balloon catheter to a first pressure, and the distal portion of the stent is expandable by application of a second force greater than the first force by inflating the balloon catheter to a second pressure.

27. The method of claim 26, wherein the proximal portion of the stent is formed with deformable elements having a smaller width than deformable elements of the distal portion of the device.

28. The method of claim 26, wherein the proximal portion of the stent is formed with deformable elements having a smaller thickness than deformable elements of the distal portion of the device.

29. The method of claim 26, wherein the proximal portion of the stent is formed of a different material than the distal portion allowing the proximal end to be deformed more easily than the distal end.

30. The method of claim 26, wherein the proximal portion of the stent is formed of a different strut configuration than the distal portion allowing the proximal portion to be deformed more easily than the distal portion.

31. The method of claim 25, wherein the stent is expanded by a first balloon catheter, the first balloon catheter is removed and a second stent is expanded in a main vessel passing across the side vessel opening of the bifurcation.

32. The method of claim 31, wherein the expansion of the second stent crushes at least a portion of the proximal end of the stent against a wall of the main vessel.

33. A bifurcation stent and delivery system comprising:
a stent comprising a continuous tubular body expandable from a delivery configuration to an expanded tissue supporting configuration, the body having a distal tubular tissue supporting portion configured to be positioned within a side branch vessel of a bifurcation and a proximal crushable portion connected to the distal tubular portion and configured to be positioned within a main vessel lumen adjacent the bifurcation, the proximal tubular having a crush strength less than the distal tubular portion; and
a balloon catheter comprising a balloon connected to a distal end of an elongated catheter shaft, the stent positioned on the balloon.

34. The stent of claim 33, wherein the distal tubular portion has an axial length equal to or greater than the proximal tubular portion.

35. The stent of claim 33, wherein the proximal tubular portion is crushable by a force which is 80% or less of a force required to crush the distal tubular portion.

36. The stent of claim 33, wherein the stent comprises a structure of substantially non-deforming struts interconnected by deformable hinges.

37. The stent of claim 36, wherein the hinges in the proximal crushable portion have a cross section less than the hinges in the distal tubular tissue supporting section.

38. The stent of claim 33, wherein the distal tubular tissue supporting portion includes a drug.

39. The stent of claim 38, wherein the proximal crushable portion does not include a drug.

40. The stent of claim 33, wherein the proximal tubular portion is crushable by a force which is 60% or less of a force required to crush the distal tubular portion.

41. The stent of claim 33, wherein the stent includes a drug and a portion of the stent corresponding to the ostium of the bifurcation includes a higher drug concentration than a remainder of the stent.
